(19) United States
(12) Patent Application Publication
(10) Pub. No.: US 2012/0095334 A1
(43) Pub. Date: Apr. 19, 2012

(54) CONTROLLED INFLATION OF AN EXPANDABLE MEMBER DURING A MEDICAL PROCEDURE

(76) Inventors: David C. Forster, Los Altos Hills, CA (US); Yenyu Chen, Palo Alto, CA (US)

(21) Appl. No.: 12/905,928

(22) Filed: Oct. 15, 2010

(51) Int. Cl. A61B 8/00; A61M 29/00

(52) U.S. Cl. 600/439; 606/192

(57) ABSTRACT

Devices and methods for controlled inflation of a vessel lumen or a hollow portion of another organ located within a patient. The devices are typically catheter-based having an expandable member fixed to a distal end of the catheter. The devices and methods typically comprise deploying the expandable member percutaneously to a target location, expanding the expandable member, and performing an expansion procedure. The expandable member expands at a controlled rate of inflation during a medical procedure.

### FIELD OF THE INVENTION

The present invention relates generally to medical devices and methods. More particularly, the present invention relates to methods and devices for controlling inflation of an expandable member in a lumen within the body during minimally invasive surgical interventions.

### BACKGROUND OF THE INVENTION

Minimally invasive surgery provides several advantages over conventional surgical procedures, including reduced recovery time, reduced surgically-induced trauma, and reduced post-surgical pain. Moreover, the expertise of surgeons performing minimally invasive surgery has increased significantly since the introduction of such techniques in the 1980s. As a result, substantial focus has been paid over the past twenty years to devices and methods for facilitating and improving minimally invasive surgical procedures.

One area in which there remains a need for substantial improvement is pre-surgical assessment of treatment locations intended to be subjected to a minimally invasive surgical procedure. For example, when a surgical procedure is to be performed at a treatment location within the body of a patient, it would frequently be beneficial for the surgeon to assess the shape, size, topography, compliance, and other physical properties of the treatment location and use the information to control devices performing procedures within a body lumen or within a hollow portion of an organ located within the body of the patient.

A particular portion of the anatomy for which complete and accurate physical assessment and control of treatment would be beneficial are the coronary valves. Diseases and other disorders of heart valves affect the proper flow of blood from the heart. Two categories of heart valve disease are stenosis and incompetence. Stenosis refers to a failure of the valve to open fully, due to stiffened valve tissue. Incompetence refers to valves that cause inefficient blood circulation, permitting backflow of blood in the heart.

Medication may be used to treat some heart valve disorders, but many cases require replacement of the native valve with a prosthetic heart valve. In such cases, a thorough assessment of the shape, size, topography, compliance, and other physical properties of the native valve annulus would be extremely beneficial. Prosthetic heart valves can be used to replace any of the native heart valves (aortic, mitral, tricuspid or pulmonary), although repair or replacement of the aortic or mitral valves is most common because they reside in the left side of the heart where pressures are the greatest.

A conventional heart valve replacement surgery involves accessing the heart in the patent's thoracic cavity through a longitudinal incision in the chest. For example, a median sternotomy requires cutting through the sternum and forcing the two opposing halves of the rib cage to be spread apart, allowing access to the thoracic cavity and heart within. The patient is then placed on cardiopulmonary bypass which involves stopping the heart to permit access to the internal chambers. After the heart has been arrested the aorta is cut open to allow access to the diseased valve for replacement. Such open heart surgery is particularly invasive and involves a lengthy and difficult recovery period.

Less invasive approaches to valve replacement have been proposed. The percutaneous implantation of a prosthetic valve is a preferred procedure because the operation is performed under local anesthesia, does not require cardiopulmonary bypass, and is less traumatic.

### BRIEF SUMMARY OF THE INVENTION

The present invention provides methods and devices for controlled inflation of a vessel lumen or a hollow portion of an organ located within a patient. The methods and devices may find use in the coronary vasculature, the atrial appendage, the peripheral vasculature, the abdominal vasculature, and in other ducts such as the biliary duct, the fallopian tubes, and similar lumen structures within the body of a patient. The methods and devices may also find use in the heart, lungs, kidneys, or other organs within the body of a patient. Moreover, although particularly adapted for use in vessels and organs found in the human body, the apparatus and methods may also find application in the treatment of animals.

The methods and devices include use of an assessment member that is preferably located at or near the distal end of a catheter or other similar device. The assessment member is introduced to a treatment location within the patient, preferably the native cardiac valve, where the assessment member is activated or otherwise put into use to perform an assessment of one or more physical parameters of the treatment location, to collect the assessment information, and to provide the assessment information to the clinician. Assessment information includes the size (e.g., diameter, circumference, area, volume, etc.) of the valve space, the shape (e.g., round, spherical, irregular, etc.) of the lumen or hollow portion of the organ, the topography (e.g., locations, sizes, and shapes of any irregular features) of the lumen or hollow portion of the organ, the nature of any regular or irregular features (e.g., thrombosis, calcification, healthy tissue, fibrosa) and the spatial orientation (e.g., absolute location relative to a fixed reference point, or directional orientation) of a point or other portion of the treatment location. Access to the treatment location is obtained by any conventional method, such as by general surgical techniques, less invasive surgical techniques, or percutaneously. A preferred method of accessing the treatment location is transluminally, preferably by well-known techniques for accessing the vasculature from a location such as the femoral artery. The catheter is preferably adapted to engage and track over a guidewire that has been previously inserted and routed to the treatment site.

The assessment mechanism includes an expandable member that is attached to the catheter shaft at or near its distal end. The expandable member may comprise an inflatable balloon, a structure containing a plurality of interconnected metallic or polymeric springs or struts, an expandable “wisk”-like structure, or other suitable expandable member. In the case of an inflatable balloon, the expandable member is operatively connected to a source of inflation medium that is accessible at or near the proximal end of the catheter. The expandable member has at least two states, an unexpanded state and an expanded state. The unexpanded state generally corresponds with delivery of the assessment mechanism through the patient's vasculature. The expanded state generally corresponds with the assessment process. The expandable member is adapted to provide assessment information to the user when the expandable member is engaged with a treatment location within the body of a patient.

Turning to several exemplary devices and methods, in one aspect of the invention, a catheter-based system includes a transluminal imaging device contained partially or entirely within an expandable structure attached at or near the distal end of the catheter.

In the preferred embodiments, the expandable member is a balloon member. The balloon member is connected to an inflation lumen that runs between the proximal and distal ends of the catheter, and that is selectively attached to a source of inflation medium at or near the proximal end of the catheter. The balloon member is thereby selectively expandable while the imaging device is located either partially or entirely within the interior of the balloon. The imaging device is adapted to be advanced, retracted, and rotated within the balloon, thereby providing for imaging in a plurality of planes and providing the ability to produce three-dimensional images of the treatment site.

In use, the transluminal imaging device is first introduced to the target location within the patient, such as the native valve annulus. In the preferred embodiment, this is achieved by introducing the catheter through the patient's vasculature to the target location. Typically, the catheter tracks over a guidewire that has been previously installed in any suitable manner. The imaging device may be provided with a radiopaque or other suitable marker at or near its distal end in order to facilitate delivery of the imaging device to the target location by fluoroscopic visualization or other suitable means. Once the imaging device is properly located at the target location, the expandable structure is expanded by introducing an expansion medium through the catheter lumen. The expandable structure expands such that it engages and applies pressure to the internal walls of the target location, such as the valve annulus. The expandable structure also takes on the shape of the internal surface of the target location, including all contours or other topography. Once the expandable structure has been sufficiently expanded, the imaging device is activated. Where appropriate, the imaging device is advanced, retracted, and/or rotated to provide sufficient movement to allow a suitable image of the target location to be created, or to collect a desired amount of measurement information. The measurement information collected and/or the images created by the imaging device are then transmitted to a suitable user interface, where they are displayed to the clinician.

In use, the expandable member is first introduced to the target location within the patient. In the preferred embodiment, this is achieved by introducing the catheter through the patient's vasculature to the target location. The catheter tracks over a guidewire that has been previously installed in any suitable manner. The expandable member carried on the catheter may be provided with a radiopaque or other suitable marker at or near its distal end in order to facilitate delivery of the physical assessment member to the target location by fluoroscopic visualization or other suitable means. Once the expandable member is properly located at the target location, the expandable member is expanded by introducing an expansion medium through the catheter lumen. The expandable member expands to a predetermined size such that the expandable member is able to engage the lumen or hollow portion of the organ, thereby providing an indicator of the shape and orientation of the lumen or hollow portion of the organ. In this way, the clinician is able to obtain precise measurements of the shape and orientation of the lumen or hollow portion of the organ at the target location. In a further preferred embodiment, the expandable member may be expanded to a size greater than the lumen or hollow portion of the organs to provide additional assessment information.

In a further aspect of the present invention, a valvuloplasty procedure is performed in association with the assessment of the native cardiac valve. In an embodiment, the expandable member also functions as a valvuloplasty balloon. The expandable member is placed within the cardiac valve space, where it is expanded. Expansion of the expandable member causes the native valve to increase in size and forces the valve, which is typically in a diseased state in which it is stiff and decreased in diameter, to open more broadly. The valvuloplasty procedure may therefore be performed prior to the deployment of a prosthetic valve, but during a single interventional procedure. In a preferred embodiment, there is controlled inflation and deflation of the expandable member used for valvuloplasty to enhance inflation and deflation rates and pressures for maximum safety and efficacy of the valvuloplasty procedure. A computer processor may be utilized to control the rate of inflation during inflation, the rate of deflation during deflation and during the valvuloplasty procedure. In a further preferred embodiment, the expandable member after performing valvuloplasty may be expanded beyond the shape and size of the native cardiac valve to distort the native cardiac valve and perform an assessment function. The advantages of controlled inflation and deflation of the expanded member may be applied to medical procedures other than valvuloplasty.

The measurement and diagnostic processes performed by any of the foregoing devices and methods may be used to facilitate any suitable medical diagnosis, treatment, or other therapeutic processes. One particular treatment that is facilitated by the foregoing devices and methods is the repair and/or replacement of coronary valves, particularly aortic valve replacement using a prosthetic valve.

Other aspects, features, and functions of the inventions described herein will become apparent by reference to the drawings and the detailed description of the preferred embodiments set forth below.

### DESCRIPTION OF THE DRAWINGS

FIG. 1 is a perspective view of a catheter in accordance with several of the embodiments of the present invention.

FIG. 2A is a cross-sectional view of an imaging device in accordance with the present invention.

FIG. 2B is a cross-sectional view of the imaging device of FIG. 2A, showing an expandable member in its expanded state.

FIG. 3 is an illustration of an exemplary apparatus for performing controlled inflation during a valvuloplasty procedure.

FIG. 4 is a graphical illustration of pressure versus balloon volume during a valvuloplasty procedure.

### DETAILED DESCRIPTION OF THE PREFERRED EMBODIMENTS

The present invention is directed to methods and devices for assessing the orientation, shape, size, topography, contours, and other aspects of anatomical vessels and organs using minimally invasive surgical techniques. As summarized above, the devices are typically catheter-based devices. Such devices are suitable for use during less invasive and minimally invasive surgical procedures. However, it should be understood that the devices and methods described herein are also suitable for use during surgical procedures that are more invasive than the preferred minimally invasive techniques described herein.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which these inventions belong. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present inventions.

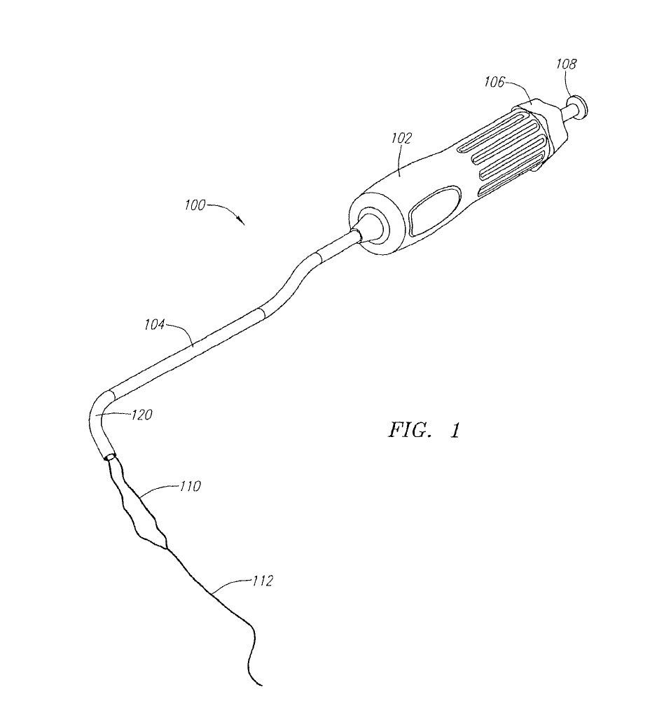

Turning to the drawings, FIG. 1 shows a catheter 100 suitable for use with assessment mechanisms described herein. The catheter 100 includes a handle 102 attached to the proximal end of an elongated catheter shaft 104. The size and shape of the handle 102 may vary, as may the features and functionality provided by the handle 102. In the illustrated embodiment, the handle 102 includes a knob 106 rotatably attached to the proximal end of the handle 102. The knob 106 may be rotated to control the movement and/or function of one or more components associated with the catheter 100, such as for retraction of one or more catheter shafts or sheaths, or manipulation of an expandable member or other component carried at or near the distal end of the catheter shaft 104. Alternative structures may be substituted for the knob 106, such as one or more sliders, ratchet mechanisms, or other suitable control mechanisms known to those skilled in the art.

An inflation port 108 is located near the proximal end of the handle 102. The inflation port 108 is operatively connected to at least one inflation lumen that extends through the catheter shaft 104 to an expandable member 110 located near the distal end of the catheter shaft 104. The inflation port 108 is of any suitable type known to those skilled in the art for engaging an appropriate mechanism for providing an inflation medium to inflate the expandable member 110.

The catheter 100 is adapted to track a guidewire 112 that has been previously implanted into a patient and routed to an appropriate treatment location. A guidewire lumen extends through at least the distal portion of the catheter shaft 104, thereby providing the catheter 100 with the ability to track the guidewire 112 to the treatment location. The catheter 100 may be provided with an over-the-wire construction, in which case the guidewire lumen extends through the entire length of the device. Alternatively, the catheter 100 may be provided with a rapid-exchange feature, in which case the guidewire lumen exits the catheter shaft 104 through an exit port at a point nearer to the distal end of the catheter shaft 104 than the proximal end thereof.

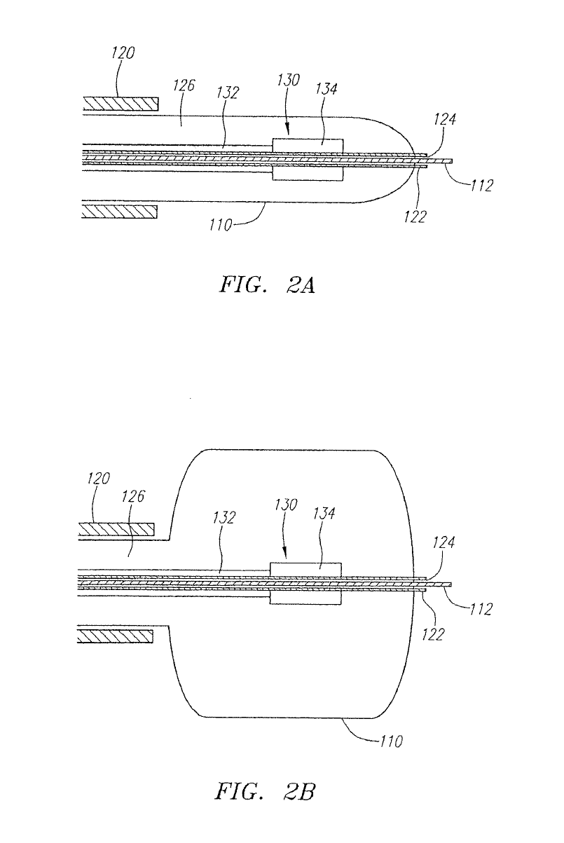

Turning next to FIGS. 2A-B, an assessment mechanism is shown and described. The assessment mechanism is located at the distal end of a catheter 100, such as that illustrated in FIG. 1 and described above. The assessment mechanism shown in FIGS. 2A-B includes an imaging device that is used to provide two-dimensional or three-dimensional images of a vessel lumen or the hollow portion of an organ within the body of a patient, as described below.

The assessment mechanism includes the outer sheath 120 of the catheter shaft 104, which surrounds the expandable member 110. In the preferred embodiment, the expandable member 110 is an inflatable balloon. The expandable member 110 is attached at its distal end to a guidewire shaft 122, which defines a guidewire lumen 124 therethrough. The guidewire 112 extends through the guidewire lumen 124.

An imaging member 130 is contained within the expandable member 110. The imaging member 130 is supported by a shaft 132 that extends proximally to the handle 102, where it is independently controlled by the user. The imaging member shaft 132 is coaxial with and surrounds the guidewire shaft 124, but is preferably movable (e.g., by sliding) independently of the guidewire shaft 124. At the distal end of the imaging member shaft 132 is the imaging head 134. The imaging head 134 may be any mechanism suitable for transmitting and receiving imaging signals. A typical imaging head 134 is an ultrasonic imaging probe for ultrasound imaging. It is within the scope of the present invention to have other imaging members 130. Such other imaging members 130 may include but not be limited to an optical fiber in conjunction with optical coherence tomography for optical imaging or an acoustic imaging device for transesophageal echo. The expandable member 110 is subject to expansion when a suitable expansion medium is injected into the expandable member through the inflation lumen 126. The inflation lumen 126, in turn, is connected to the inflation port 108 associated with the handle 102. FIG. 2A illustrates the expandable member 110 in its unexpanded (contracted) state, while FIG. 2B illustrates the expandable member 110 in its expanded state, such as after a suitable inflation medium is injected through the inflation port 108 and inflation lumen 126 into the expandable member 110.

To use the assessment mechanism illustrated in FIGS. 2A-B, the distal portion of the catheter is delivered to a treatment location within the body of a patient over the previously deployed guidewire 112. In a particularly preferred embodiment, the treatment location is the aortic heart valve, and the guidewire 112 is deployed through the patient's vasculature from an entry point in the femoral artery using, for example, the Seldinger technique. Deployment of the assessment mechanism is preferably monitored using fluoroscopy or other suitable visualization mechanism. Upon encountering the treatment location, the expandable member 110 is expanded by inflating the balloon with a suitable inflation medium through the inflation port 108 and the inflation lumen 126. The expandable member 110 engages the internal surfaces of the treatment location, such as the annular root of the aortic heart valve. Once the expandable member 110 is expanded, the imaging head 134 is activated and the imaging process is initiated. The imaging head 134 is preferably advanced, retracted, and rotated within the expandable member 110 as needed to obtain images in a variety of planes to yield a 360° three-dimensional image, or any desired portion thereof. Once the imaging process is completed, the expandable member 110 is deflated, and the assessment mechanism may be retracted within the catheter shaft 104. The catheter 100 is then removed from the patient.

In an exemplary embodiment, a valvuloplasty procedure is performed wherein the expandable member is placed within the cardiac valve space, where it is expanded. Expansion of the expandable member causes the native valve to increase in size and forces the valve, which is typically in a diseased state in which it is stiff and, decreased in diameter, to open more broadly.

However, over dilatation of a valvuloplasty expandable member that has a maximum diameter greater than the safe diameter of the aorta can result in injury to the patient. One such type of injury is called an aortic dissection which is when the expandable member over extends the anatomy of the aorta and the aortic wall tears causing the dissection.

While trying to avoid injury, an effective valvuloplasty result is also critical. Acceptable results vary from physician to physician but are usually considered effective if the aortic valve area is approximately doubled after valvuloplasty.

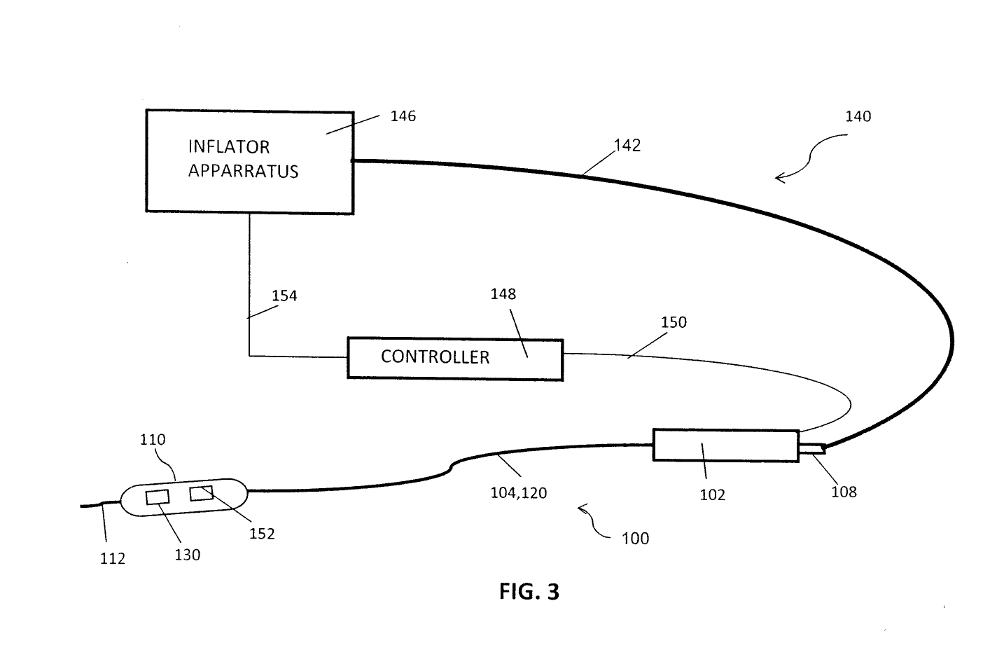

Referring now to FIG. 3, there is shown a device for controlled inflation of an expandable member during a valvuloplasty procedure. As described in FIG. 1, there is a catheter 100 which includes a handle 102, catheter shaft 104, outer sheath 120, expandable member 110 and guide wire 112. The handle 102 has an inflation port 108.

Connected to the catheter 100 is an inflation apparatus 140 which includes a tube 142 for carrying an inflation medium (not shown). Connected to the tube 142 is an inflator apparatus 146 which may be a pump to cause the inflation medium to flow through tube 142 and catheter 100, eventually ending up in expandable member 110 to cause the expandable member 110 to expand to perform a medical procedure, including but not limited to a valvuloplasty procedure. Inflator apparatus 146 may include a metering device to control the flow of inflation medium. Inflator apparatus 146 may also include a volume control to measure the amount of inflation medium passing through the tube 142. After the medical procedure has been completed, the inflator apparatus 146 reverses the flow of the inflation medium to cause the expandable member 110 to deflate. Controlling the inflator apparatus 146 is controller 148. Controller 148 may be connected to inflator apparatus 146 by wire 154 or may communicate wirelessly with inflator apparatus 146. It is within the scope of the present invention for controller 148 to be incorporated into inflator apparatus 146.

Controller 148 may be a computer, computer processor or microprocessor and may include random access memory (RAM), read-only memory (ROM) and a storage device of some type such as a hard disk drive, floppy disk drive, CD-ROM drive, tape drive or other storage device. Controller 148 may also include communication links to provide communication to other devices such as another computer.

Catheter 100 may also include an assessment mechanism as described previously. One such assessment mechanism is an imaging member 130 as described previously which may assist in determining the size, shape and orientation of the expandable member 110 in real time. Other assessment mechanisms may be present such as pressure sensors (for example, a pressure transducer) to measure the pressure in the expandable member 110. For purposes of illustration and not limitation, pressure sensor 152 is shown within expandable member 110. Pressure sensor 152 may also be outside of expandable member 110. Pressure sensor 152 may also be outside of the patient's body, such as on or near catheter handle 102.

The assessment mechanisms provide feedback to controller 148. For this purpose, wire 150 extends from handle 102 of the catheter 100 to the controller 148. Wire 150 may extend up into expandable member 110 to relay information from imaging member 130 and pressure sensor 152 to controller 148. It is within the scope of the present invention for imaging member 130 and pressure sensor 152 to communicate wirelessly with controller 148.

Based on the feedback provided to controller 148 from the assessment mechanisms, controller 148 controls inflator apparatus to vary the rate and the extent of inflation and deflation of the expandable member 110. In one exemplary embodiment, the expandable member may be only partially deflated.

In a preferred embodiment, pressure information and volume information of the expandable member are fed back to controller 148. Pressure information may be obtained from a pressure sensor, for example, while volume information may be obtained from the metering device or volume control which may be located in the inflator apparatus 146. The controller 148 uses an algorithm that tracks inflation pressure, inflation volume and aortic tissue anatomical changes resulting from the change in pressure and volume and in turn controls the inflator apparatus 146 to control the inflation of the expandable member 110.

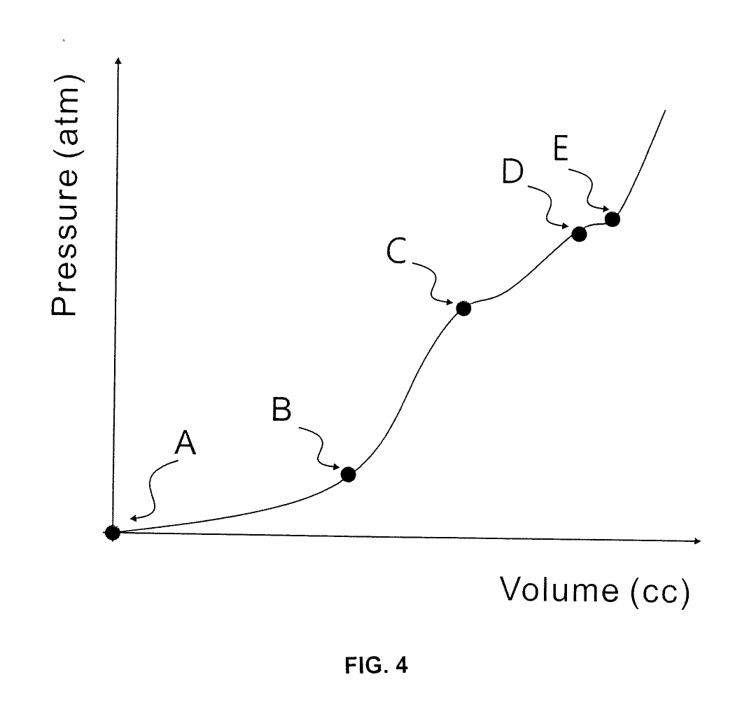

FIG. 4 is a graphical illustration of a valvuloplasty procedure. Starting at point A in FIG. 4, there is no pressure and little volume in the expandable member 110. The inflator apparatus 146 as directed by the controller 148 causes the expandable member 110 to expand until the expandable member 110 contacts the aortic wall at point B. In the interval from point A to point B, there is little increase in pressure as the expandable member 110 expands without resistance and the line from point A to point B is steady. Until the expandable member 110 contacts the aortic wall at point B, the expandable member 110 may be expanded quite rapidly.

When the expandable member 110 comes in contact with the aortic wall, the expandable member 110 begins to push the material making up the aortic root/annulus, thus performing valvuloplasty. Increased volume and pressure are required to achieve an effective clinical result. Referring again to FIG. 4, calcium fracturing may occur at point C and again at point D.

At point D shown in FIG. 4, all modes of valvuloplasty may have been completed.

The various changes in pressure and volume in the interval from point B to point D are fed back to the controller 148 and analyzed there. The interval from point B to point D is characterized as an unsteady rise in pressure and volume. With such characterization, the controller 148 knows that there is valvuloplasty occurring and slows down the rate of inflation of the expandable member 110.

At point E in FIG. 4, there is rapid increase in pressure with little increase in volume which validates that valvuloplasty is completed and further expansion of the expandable member 110 could cause tearing of the aortic wall. The controller, having received this latest pressure and volume assessment information, halts the expanding of the expandable member 110 and then begins the deflation and subsequent withdrawal of the expandable member 110.

The imaging member 130 within expandable member 110 may also assist with assessment information in determining when the expandable member 110 has contacted the aortic wall and the increased expansion of the aortic wall.

The preferred embodiments of the inventions that are the subject of this application are described above in detail for the purpose of setting forth a complete disclosure and for the sake of explanation and clarity. Those skilled in the art will envision other modifications within the scope and spirit of the present disclosure. Such alternatives, additions, modifications, and improvements may be made without departing from the scope of the present inventions, which is defined by the claims.

What is claimed is:

1. A device for controlled inflation of an expandable member during a medical procedure comprising:
an inflation apparatus comprising an inflation device, an expandable member and an inflation lumen connecting the inflation device and the expandable member;
at least one assessment mechanism to provide assessment information; and
a controller in communication with the assessment mechanism to receive the assessment information and with the inflation device, the controller controlling the inflation device in response to the assessment information received to cause controlled inflation and deflation of the expandable member.

2. The device of claim 1 wherein the assessment information comprises pressure and volume information of the expandable member.

3. The device of claim 1 wherein the assessment information comprises size, shape and orientation of the expandable member.

4. The device of claim 2 wherein the assessment information comprises size, shape and orientation of the expandable member.

5. The device of claim 1 wherein the assessment mechanism comprises an imaging device to view the expandable member during controlled inflation and deflation.

6. The device of claim 5 wherein the imaging device is an optical imaging device.

7. The device of claim 5 wherein the imaging device is an ultrasound imaging device.

8. The device of claim 1 wherein the expandable member is a balloon.

9. The device of claim 1 wherein the assessment mechanism is located within the expandable member.

10. The device of claim 1 wherein the assessment mechanism is located outside the expandable member.

11. The device of claim 1 wherein the medical procedure is a valvuloplasty.

12. A method for controlled inflation of an expandable member comprising the steps of:
expanding the expandable member at a first rate controlled by a controller until the expandable member contacts a wall of a lumen;
expanding the expandable member at a second rate controlled by a controller during a medical procedure wherein the second rate is slower than the first rate; and
halting expanding of the expandable member when the medical procedure is complete as determined by a controller.

13. The method of claim 12 wherein the expandable member is a balloon.

14. The method of claim 12 further comprising deflating the expandable member after each step of expanding.

15. The method of claim 14 further comprising imaging the expandable member during the steps of expanding and deflation.

16. The method of claim 15 wherein the imaging is by optical imaging.

17. The method of claim 15 wherein the imaging is by ultrasound imaging.

18. The method of claim 12 wherein the medical procedure is valvuloplasty and the lumen is a cardiac valve.

19. A method for controlled inflation of an expandable member comprising the steps of:
deploying an expandable member within the body of a patient;
assessing an expandable member at frequent intervals to determine assessment information;
providing the assessment information to a controller;
expanding the expandable member at a first rate until the expandable member contacts a wall of a lumen, the first rate determined by the controller responsive to assessment information received;
expanding the expandable member at a second rate during a medical procedure wherein the second rate is slower than the first rate, the second rate determined by the controller responsive to assessment information received;
halting expanding of the expandable member when the medical procedure is complete as determined by the controller responsive to assessment information received.

20. The method of claim 19 wherein the assessment information comprises pressure and volume information of the expandable member.

21. The method of claim 19 wherein the assessment information comprises size, shape and orientation of the expandable member.

22. The method of claim 20 wherein the assessment information comprises size, shape and orientation of the expandable member.

23. The method of claim 19 further comprising deflating the expandable member after each step of expanding.

24. The method of claim 23 wherein the step of assessing is by an imaging device to view the expandable member during each step of expanding and deflation.

25. The method of claim 24 wherein the imaging device is an optical imaging device.

26. The method of claim 24 wherein the imaging device is an ultrasound imaging device.

27. The method of claim 19 wherein the expandable member is a balloon.

28. The method of claim 19 wherein the medical procedure is valvuloplasty and the lumen is a cardiac valve.
